(19) United States
(12) Patent Application Publication
(10) Pub. No.: US 2010/0148090 A1
(43) Pub. Date: Jun. 17, 2010

(54) MULTI-PURPOSE ULTRAVIOLET STERILIZER

(76) Inventor: CHWEN-CHWEN CHANG, Taipei (TW)

Correspondence Address: SCHMEISER OLSEN & WATTS, 18 E UNIVERSITY DRIVE, SUITE # 101, MESA, AZ, 85201, US

(21) Appl. No.: 12/334,839

(22) Filed: Dec. 15, 2008

(51) Int. Cl. G01N 23/00; A61L 2/10; A61L 2/07

(52) U.S. Cl. 250/455.11; 422/300; 422/291

(57) ABSTRACT

A multi-purpose ultraviolet sterilizer comprises a body having six planes, wherein one of the six planes is a door; an inner space arranged inside the body and used to store objects that are small the inner space; and a sterilization module coupled with the inner space and used to disinfect objects stored inside the inner space.

### BACKGROUND OF THE INVENTION

1. Field of the Invention

The present invention relates to a disinfection and storage structure, particularly to a multi-purpose ultraviolet sterilizer.

2. Description of the Related Art

A person usually has to store personal belongings, including jewelry (such as necklaces, earrings, and other ornaments), precious items (such as watches), photo equipments (such as cameras and lenses), 3C products, foods, and various types of utensils. The personal belongings, foods, utensils or equipments need periodic disinfection or preserve in an environment of a specified temperature and humidity, which may require a storage device that performs disinfection and temperature/humidity control. Therefore, a disinfection storage box that has all the abovementioned functions is in great demand.

As a result, the present invention proposes a multi-purpose ultraviolet sterilizer that allows users to conveniently disinfect and store personal belongings, foods, utensils and/or equipments to satisfy consumers.

### SUMMARY OF THE INVENTION

One embodiment of the present invention provides a multi-purpose ultraviolet sterilizer, which comprises a body having six planes with one of the six planes being a door; an inner space allotted inside the body and used to store objects smaller than the inner space, wherein the door is opened to place objects into the inner space; and a sterilization module coupled with the inner space to disinfect objects stored inside the inner space.

Some of the features and advantages of the present invention are to be described below. Some other features and advantages of the present invention should be obvious for the persons skilled in the art from the following description, and some other features and advantages will be self-evident in the application of the present invention, although they are not directly stated thereinafter. In addition, realizing the claims stated below will embody the features and advantages of the present invention.

It should be noted that the foregoing schematic description and the following detailed description of the present invention is only to exemplify the present invention but not to limit the scope of the present invention.

### BRIEF DESCRIPTION OF THE DRAWINGS

FIG. 1 is a diagram schematically showing a multi-purpose ultraviolet sterilizer according to one embodiment of the present invention;

FIG. 2 is an exploded view of a multi-purpose ultraviolet sterilizer according to one embodiment of the present invention;

FIG. 3 is a diagram schematically showing the operation of a steam sterilization module of a multi-purpose ultraviolet sterilizer according to one embodiment of the present invention;

FIG. 4 is a diagram schematically showing the operation of a steam sterilization module and a freezing sterilization module of a multi-purpose ultraviolet sterilizer according to one embodiment of the present invention; and

FIG. 5 is a diagram schematically showing the operation of an ultraviolet sterilization module of a multi-purpose ultraviolet sterilizer according to one embodiment of the present invention.

### DETAILED DESCRIPTION OF THE INVENTION

Below, the embodiments of the present invention are described in cooperation with the attached drawings. An identical numeral used in the drawings should direct to the identical or similar elements in the drawings.

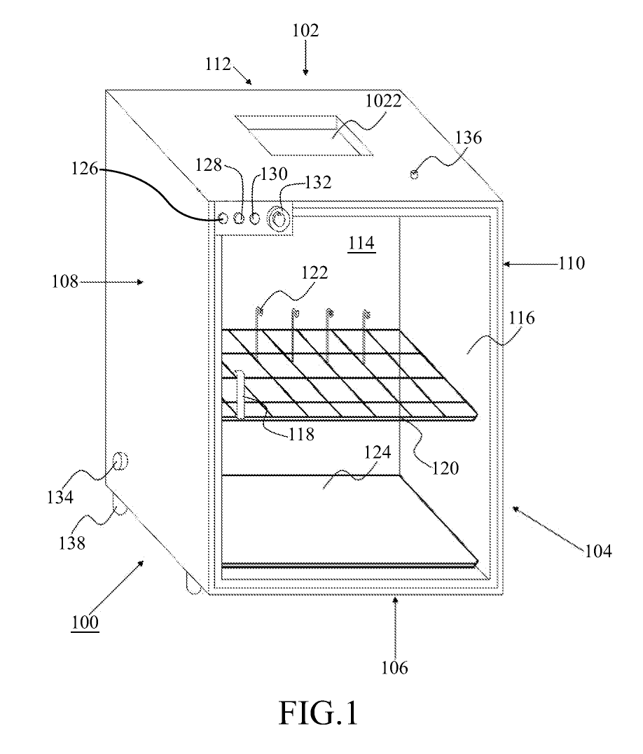

Referring to FIG. 1, a diagram schematically shows a multi-purpose ultraviolet sterilizer according to one embodiment of the present invention. The multi-purpose ultraviolet sterilizer 100 of the present invention comprises a body, an inner space 114 and a sterilization module. The body has a top plane 102, a first plane 104, a bottom plane 106, a second plane 108, a third plane 110, and a fourth plane 112. The first plane 104 is a door 116. In one embodiment, the door 116 has a handle 118. The inner space 114 is arranged inside the body, and the sterilization module is coupled with the inner space 114. The inner space 114 is used to store the objects smaller than the inner space 114. A user can hold the handle 118 to open the door 116 and place objects into the inner space 114. The sterilization module is used to disinfect the objects stored in the inner space 114.

The inner space 114 may have a separation plate 120 to store different types of objects. The separation plate 120 may be a platform, a mesh or a grille. The separation plate 120 may further have a hook 112 to hang an object. One embodiment has a plurality of separation plates 120, which may be platforms, meshes or grilles. The plurality of separation plates 120 may further have a plurality of hooks 122 to hang objects. The top plane 102 may further have a basin 1022 used to store objects smaller than the basin 1022. In one embodiment, the top plane 102 has a plurality of basins 1022.

It should be noted that the door 116 may be arranged on one of the planes of the body except the bottom plane 106. The door 116 is made of a transparent material or an opaque material. The bottom plane 106 has several legs 138 to support the body. In one embodiment, the bottom plane 106 further has a storage space, which may a detachable box or a detachable drawer. It should be understood that other storage structures installed on the bottom plane 106 are also included within the scope of the present invention.

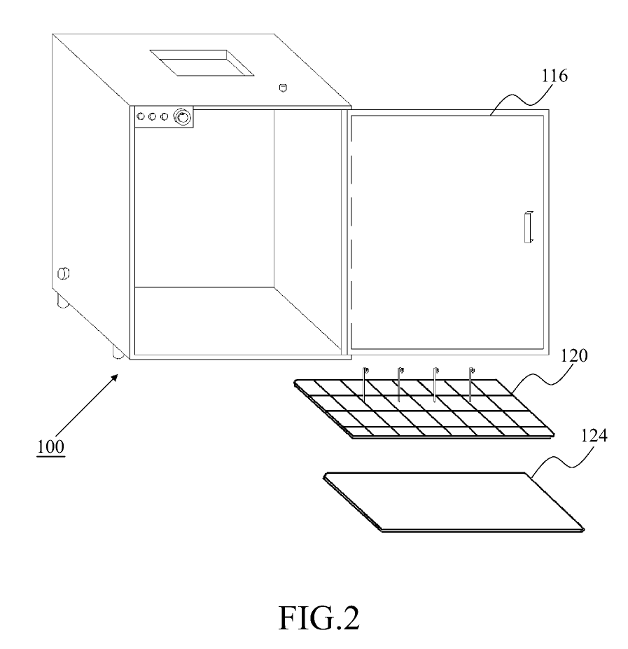

Referring to FIG. 2, an exploded view of a multi-purpose ultraviolet sterilizer according to one embodiment of the present invention is shown. As shown in FIG. 2, after the door 116 is opened, the separation plates 120 and 124 can be detached from the body for cleaning the plates or loading objects on the plates.

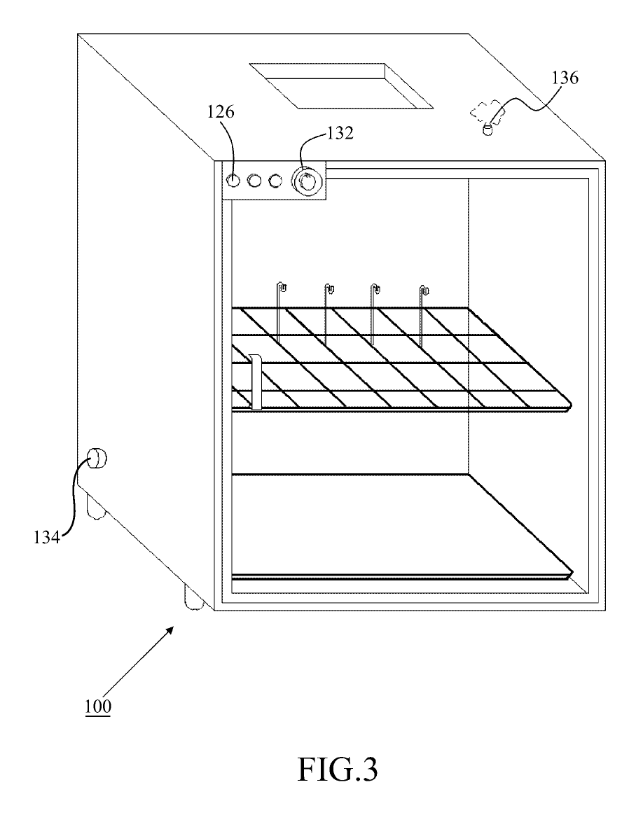

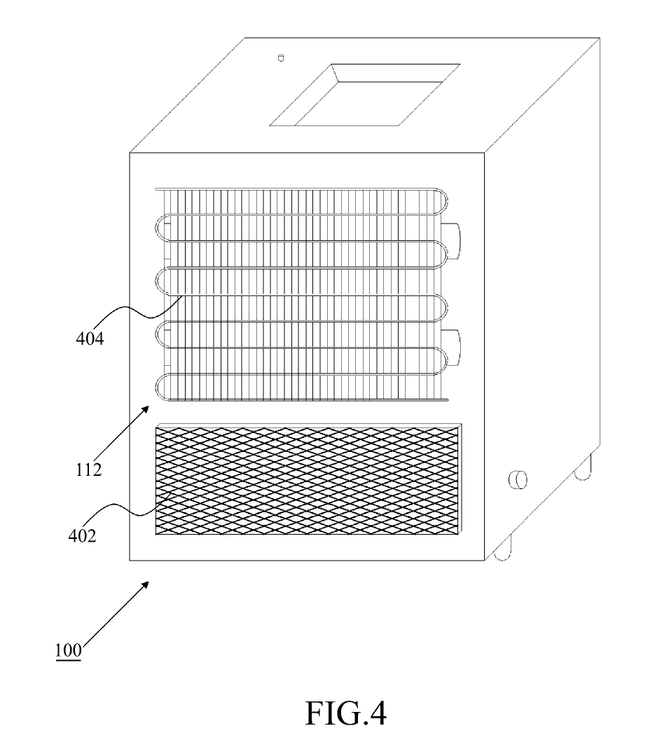

Referring to FIG. 3, a diagram schematically shows the operation of a steam sterilization module of a multi-purpose ultraviolet sterilizer according to one embodiment of the present invention. The steam sterilization module is used to heat air inside the inner space 114 to produce steam disinfecting the objects inside the inner space 114. The steam sterilization module uses a wet heat sterilization method to disinfect the objects inside the inner space 114. One of the six planes of the body has an air inlet 134 allowing air to enter the inner space, and the air is heated into steam by a heating module 402 as shown in FIG. 4. In one embodiment, the air inlet 134 is formed on the second plane 108 as shown in FIG. 1. One of the six planes of the body has an air outlet 136 used to release at least a part of or all of steam. In one embodiment, the air outlet 136 is formed on the top plane 102. The body has a first switch 126 used to turn on or turn off the steam sterilization module. The body also has a temperature control knob 132, which is used to regulate temperature of the inner space 114. It is obvious for the persons skilled in the art that the steam sterilization module can use steam to heat or keep warm objects inside the inner space 114.

Referring to FIG. 4, a diagram schematically shows the operation of a steam sterilization module and a freezing sterilization module of a multi-purpose ultraviolet sterilizer according to one embodiment of the present invention. The sterilization module may include a freezing sterilization module, which reduces the temperature of the inner space 114 to disinfect objects inside said inner space 114. The body has a second switch 128 used to turn on or turn off the freezing sterilization module. The temperature control knob 132 is used to regulate the temperature of the inner space 114. It is obvious for the persons skilled in the art that the freezing sterilization module can preserve or keep fresh objects inside the inner space 114 via lowering the temperature of the inner space 114.

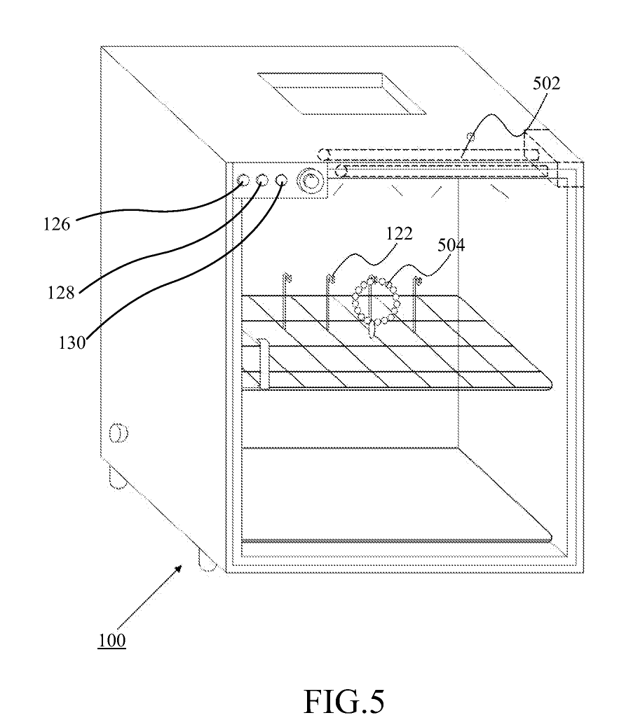

Referring to FIG. 5, a diagram schematically shows the operation of an ultraviolet sterilization module of a multi-purpose ultraviolet sterilizer according to one embodiment of the present invention. The sterilization module may include an ultraviolet sterilization module 502 using ultraviolet ray to disinfect an object 504 inside the inner space 114. The body has a third switch 130 used to turn on or turn off the ultraviolet sterilization module 502.

It should be understood that the embodiments described above are only to exemplify the present invention but not to limit the scope of the present invention, and that any equivalent modification or variation according to the spirit of the present invention is to be also included within the scope of the present invention.

The steps of the method/process of the present invention are described in a specified sequence. If the method/process does not depend on the specified sequence of the steps, the present invention should not be confined to the specified sequence. Any equivalent modification or variation of the method/process according to the spirit of the present invention is to be also included with the scope of the present invention.

What is claimed is:

1. A multi-purpose ultraviolet sterilizer comprising
a body having six planes, wherein one of said six planes is a door;
an inner space allotted inside said body and used to store objects smaller than said inner space, wherein said door is opened to place objects into said inner space; and
a sterilization module coupled with said inner space to disinfect objects stored inside said inner space.

2. The multi-purpose ultraviolet sterilizer according to claim 1, wherein said inner space further comprises at least one separation plate used to store objects.

3. The multi-purpose ultraviolet sterilizer according to claim 2, wherein said separation plate is equipped with a hook used to hang objects.

4. The multi-purpose ultraviolet sterilizer according to claim 1, wherein a top plane of said six planes has at least one basin used to accommodate objects that are smaller than said basin.

5. The multi-purpose ultraviolet sterilizer according to claim 1, wherein said sterilization module is equipped with a steam sterilization module used to heat air inside said inner space to produce steam disinfecting objects inside said inner space.

6. The multi-purpose ultraviolet sterilizer according to claim 5, wherein said steam sterilization module disinfects objects in said inner space with a wet heat sterilization method.

7. The multi-purpose ultraviolet sterilizer according to claim 5, wherein one of said six planes of said body has an air inlet allowing air to enter said inner space, and said air is heated into said steam in said inner space.

8. The multi-purpose ultraviolet sterilizer according to claim 5, wherein one of said six planes of said body has an air outlet used to release at least a part of or all of said steam.

9. The multi-purpose ultraviolet sterilizer according to claim 5, wherein said body has a first switch used to turn on or turn off said steam sterilization module.

10. The multi-purpose ultraviolet sterilizer according to claim 5, wherein said body has a temperature control knob used to regulate temperature of said inner space.

11. The multi-purpose ultraviolet sterilizer according to claim 5, wherein said steam sterilization module uses said steam to heat or keep warm objects inside said inner space.

12. The multi-purpose ultraviolet sterilizer according to claim 1, wherein said sterilization module has a freezing sterilization module, which reduces temperature of said inner space to disinfect objects inside said inner space.

13. The multi-purpose ultraviolet sterilizer according to claim 12, wherein said body has a second switch used to turn on or turn off said freezing sterilization module.

14. The multi-purpose ultraviolet sterilizer according to claim 12, wherein said body has a temperature control knob used to regulate temperature of said inner space.

15. The multi-purpose ultraviolet sterilizer according to claim 12, wherein said freezing sterilization module preserves or keeps fresh objects inside said inner space via lowering temperature of said inner space.

16. The multi-purpose ultraviolet sterilizer according to claim 1, wherein said sterilization module has an ultraviolet sterilization module using ultraviolet ray to disinfect objects inside said inner space.

17. The multi-purpose ultraviolet sterilizer according to claim 16, wherein said body has a third switch used to turn on or turn off said ultraviolet sterilization module.
